(19) United States
(12) Patent Application Publication
(10) Pub. No.: US 2022/0041989 A1
(43) Pub. Date: Feb. 10, 2022

(54) CELLULAR CULTURE MEDIUM FREE FROM SERUM

(71) Applicant: Blast Research S.r.l., Milano (IT)

(72) Inventors: Massimo Costa, Milano (IT); Alberto Giuseppe Passi, Milano (IT)

(73) Assignee: Blast Research S.r.l., Milano (IT)

(21) Appl. No.: 17/505,688

(22) Filed: Oct. 20, 2021

Related U.S. Application Data
(63) Continuation of application No. 15/749,183, filed on Jan. 31, 2018.

(30) Foreign Application Priority Data
Aug. 3, 2015 (IT) 102015000041440

(51) Int. Cl. C12N 5/077; A01N 1/02

(52) U.S. Cl. CPC C12N 5/0656; A01N 1/0226; C12N 2500/14; C12N 2500/95; C12N 2500/34; C12N 2500/38; C12N 2500/92; C12N 2500/16

(57) ABSTRACT

The present invention relates to a cell culture medium, in particular for culturing autologous fibroblasts, for use in aesthetic medicine for skin transplantation, said culture medium being serum-free and being characterized in that it contains glucose in a much lower quantity than conventional culture media.

### FIELD OF INVENTION

The present invention relates to a cell culture medium, especially for culturing human autologous fibroblasts intended for skin transplantation into the field of aesthetics or regenerative medicine.

### BACKGROUND ART

In aesthetic and regenerative medicine, the use of autologous fibroblasts transplantation, in jargon also called “Autotransplantation”, is a technique employed for quite some time.

With aging, the number of active fibroblasts in the skin tends to decrease, as well as the metabolic capabilities thereof. The increase of cells due to transplantation of autologous fibroblasts with the ability to synthesize the extracellular matrix allows for counteracting the effects of skin aging.

Indeed, it was clinically demonstrated that, in patients who were injected with cultures of autologous fibroblasts from explants of the same patients and cultivated in vitro, they have improved the biomechanical properties of skin of above-mentioned patients, as demonstrated with different kinds of clinical trials, such as optical profilometry, vacuum cutometry, and the VISIA® photometric analysis.

Indeed, the fibroblasts injected were able to synthesize the extracellular matrix for at least 12 months.

In areas of the skin of the patients who have undergone treatment actually, it is noted an increase of collagen and a general thickening of dermis (up to 60% in 12 months), an increase of elasticity of the skin (25% in the periorbital area) and a general reduction of wrinkles equal to 50% (V. Zorin et al “Clinical-instrumental and morphological evaluation of the effect of autologous dermal fibroblasts administration” Journal of Tissue Engineering and Regenerative Medicine Article first published online: 19 Dec. 2014b DOI: 10.1002/term.1976).

Although the good results obtained with this technique, it finds it hard to take hold for a number of criticalities, first of all the cell culture medium.

In fact, it is well known that the culture media in which cells such as fibroblasts grows are not always suitable to be injected into humans.

In fact, it is known that the cells exhibit an excellent cell viability and proliferation in the presence of serum and the serum generally employed is the fetal bovine serum (PBS). This type of substances can be immunogen and therefore cause allergic reactions in some subjects. Other types of cell culture media such as DMEM have extremely high amounts of glucose that can be dangerous in subjects with overt diabetes or with diabetic tendencies.

It's felt the need to have cell culture media which do not present the aforesaid drawbacks, i.e. that are serum-free while containing a lower content of glucose, compared to the cell culture media such as DMEM.

On the other hand, it is known a physiological saline solution, called Tyrode's solution, which allows for storing organs for transplantation at low temperatures for 10-12 hours.

This aqueous solution consists of the following components in the respective concentrations

Standard Tyrode's solution

| | | |
|---|---|---|
| NaCI | 137 | mM |
| KCl | 2.7 | mM |
| D-Glucose | 5.5 | mM |
| NaHCO₃ | 12 | mM |
| NaH₂PO₄ | 0.2 | mM |
| MgCl₂ | 1 | mM |
| CaCl₂ | 1.8 | mM |

### SUMMARY OF THE INVENTION

The applicant has now found a saline solution, which, when employed as cell culture medium, in particular for culturing fibroblasts, maintains up to 30% of viable cells for a 24 hours' incubation period in said liquid. Also this type of saline solution allows for a markedly higher cell growth than a culture medium containing only PBS.

Therefore, an object of the present invention is an aqueous saline solution containing

a) a mixture of mineral salts consisting of sodium chloride, potassium chloride, sodium bicarbonate, sodium dihydrogen phosphate, magnesium chloride and calcium chloride,

b) D-glucose and

c) at least a water-soluble vitamin,

wherein the concentration of b) D-glucose is <6 mM.

A further object of the present invention is a serum-free cell culture medium consisting of the above-mentioned physiological solution.

Finally, a further object of the present invention is a cell culture of human autologous fibroblasts in the culture medium according to the present invention for use in skin transplantation in aesthetic or regenerative medicine.

### DESCRIPTION OF FIGURES

FIG. 1 shows in graphical presentation the MTT assay results on % cell viability of fibroblasts after 24 hours of incubation in DMEM, DMEM+PBS, PBS only, conventional Tyrode's solution, a solution according to the present invention in the presence of Vitamin C (0.1 mM) and Vitamin B6 (10 mM) and a solution according to the present invention in the presence of Vitamin C (1 mM), and Vitamin B6 (10 mM), respectively.

FIG. 2 shows proliferation of human fibroblasts (cell counting) after a 24 hours' incubation in the following culture media: DMEM only, DMEM+PBS, PBS only, conventional Tyrode's solution, a solution according to the present invention in the presence of Vitamin C (0.1 mM), Vitamin B6 (10 mM) and a solution according to the present invention in the presence of Vitamin C (1 mM), Vitamin B6 (10 mM).

### DETAILED DESCRIPTION OF THE INVENTION

Preferably the saline solution of the invention has a concentration of the component b), i.e. D-glucose, of 5.5 mM.

Preferably the water-soluble vitamins of the present invention are selected from at least one of the following: Vitamin C, Vitamin B12, Vitamin B6, Vitamin B5 or Pantothenic Acid, Biotin, Nicotinamide or the amide form of Vitamin B3, the salt form of α-Lipoic Acid or Vitamin N.

Preferably the saline solution of the invention contains at least the combination of Vitamin C and Vitamin B6.

Preferably for the objects of the present invention by Vitamin B6 it is intended pyridoxine, pyridoxamine hydrochloride, pyridoxal o pyridoxal phosphate.

Preferably, the Vitamin C is present at a concentration between 0.05 mM and 2 mM and the Vitamin B6 is present at a concentration ranging between 5 mM and 20 mM.

According to a particularly preferred embodiment of the invention, the saline solution of the present invention consists of the following components in their respective millimolar concentrations:

| | | |
|---|---|---|
| NaCl | 130 | mM, |
| KCl | 2.68 | mM, |
| D-Glucose | 5.5 | mM, |
| NaHCO₃ | 11.9 | mM, |
| NaH₂PO₄ | 0.46 | mM, |
| MgCl₂ | 1.05 | mM, |
| CaCl₂ | 1.8 | mM, |
| Vitamin C | 0.1-1 | mM, |
| Vitamin B6 | 10 | mM. |

Experimental evidences of cell viability and cellular proliferation of fibroblasts carried on cell cultures in DMEM, DMEM+PBS, only PBS, conventional Tyrode's solution, a solution according to the present invention in the presence of Vitamin C (0.1 mM), Vitamin B6 (10 mM) and a solution according to the present invention in the presence of Vitamin C (1 mM), and Vitamin B6 (10 mM), respectively, are reported for illustrative purposes herein below.

### Example

Method

The cells (primary human dermal fibroblasts) were grown in standard amplification conditions (37° C., 5% CO₂, 95% humidity in the incubator) in the following culture liquids
DMEM
DMEM supplemented with antibiotics and 10% serum, in the culture liquid containing PBS serum (10%)
Standard Tyrode
Two Tyrode's solutions modified according to the invention and containing each Vitamin B6 at a concentration of 10 mM and Vitamin C at a concentration of 0.1 and 1 mM, respectively.

Conventional or standard Tyrode's formulations and modified Tyrode's formulations according to the present invention and examined in the present example are shown in the following table herein below.

| | Tyrode's solution | | | |
|---|---|---|---|---|
| | modified according to | | Standard | |
| Component | the present invention | | Tyrode | |
| NaCI | 130 | mM | 137 | mM |
| KCl | 2.68 | mM | 2.7 | mM |
| D-Glucose | 5.5 | mM | 5.5 | mM |
| NaHCO3 | 11.9 | mM | 12 | mM |
| NaH2PO4 | 0.46 | mM | 0.2 | mM |
| MgCl2 | 1.05 | mM | 1 | mM |
| CaCl2 | 1.8 | mM | 1.8 | mM |

| Vit C | 0.1 mM or 1 mM | | Absent |
|---|---|---|---|
| Vit B6 | 10 | mM | Absent |

At the time of the experiment, the cells, detached by trypsin treatment and after centrifugation and washing in sterile buffer (PBS), were suspended in the various solutions prepared and then counted with an automatic cell counter.

During the 24 h incubation, the cells were maintained in the incubator as for the amplification step.

The cell cultures were then assessed for viability (MTT assay) and proliferation (cell counting).

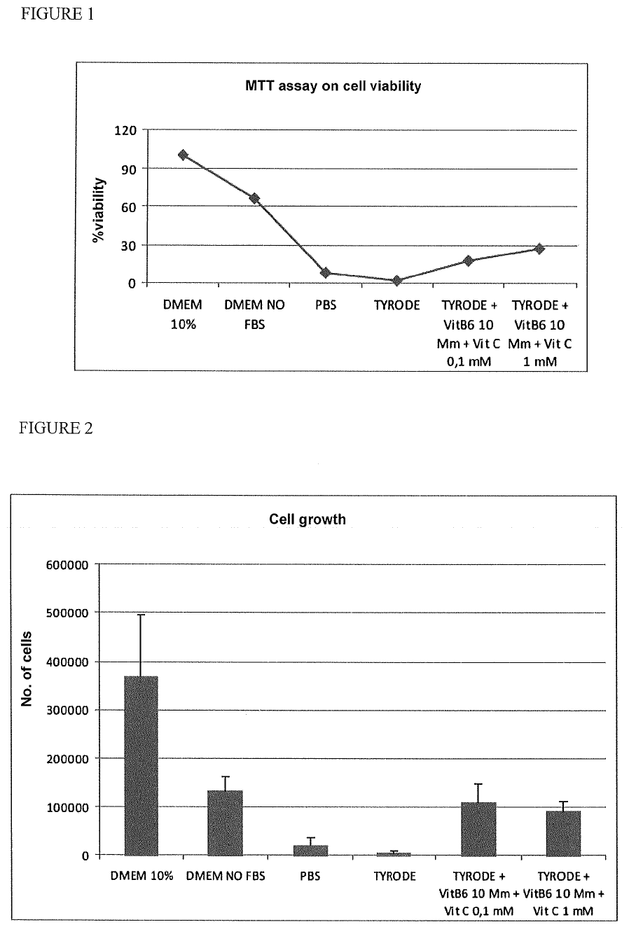

The results of the above tests are reported in FIGS. 1 and 2, respectively.

As can be seen after a 24 hours' incubation in serum-free media (PBS and Tyrode's standard) the cells have in fact completely lost their viability. In the case of culture medium DMEM with serum, the cells have more than 90% of survival that drops to just over 60% in DMEM without serum, however in the presence of high doses of amino acids, vitamins, and 25 mM glucose, which is five times the standard concentration. The modified Tyrode's solutions according to the present invention instead maintain the cells viable up to 30% after 24 hours, a much better result than the standard Tyrode's solution, but also than the culture liquid containing PBS only.

The two modified Tyrode's solution, object of the present invention and examined, show a significantly better proliferation compared to standard formulation containing PBS only and the standard Tyrode, although obviously lower than DMEM formulations that are markedly much more rich in amino acids and micronutrients.

1-9. (canceled)

10. A method for improving biomechanical properties of skin comprising transplanting to a subject in need thereof a cell culture of fibroblasts expanded in a saline aqueous solution consisting of:
a mixture of biologically acceptable mineral salts consisting of sodium chloride, potassium chloride, sodium bicarbonate, sodium dihydrogen phosphate, magnesium chloride and calcium chloride,
D-glucose,
Vitamin B6 and vitamin C, and
water;
wherein in the saline aqueous solution glucose is in a concentration<6 mM, vitamin C is in a concentration of between 0.05 mM and 2 mM and vitamin B6 is in a concentration of between 5 mM and 20 mM.

11. The method of claim 10, wherein in the saline aqueous solution the concentration of glucose is 5.5 mM.

12. The method according to claim 10, wherein said aqueous solution is a modified Tyrode's solution and consists of the following components in the respective millimolar concentrations:

| | | |
|---|---|---|
| NaCl | 130 | mM, |
| KCl | 2.68 | mM, |
| D-Glucose | 5.5 | mM, |
| NaHCO3 | 11.9 | mM, |
| NaH2PO4 | 0.46 | mM, |
| MgCl2 | 1.05 | mM, |
| CaCl2 | 1.8 | mM, |
| Vitamin C | 0.1-1 | mM, and |
| Vitamin B6 | 10 | mM. |
